(19) United States
(12) Patent Application Publication
(10) Pub. No.: US 2014/0166024 A1
(43) Pub. Date: Jun. 19, 2014

(54) MOUTHGUARD FOR THE DELIVERY OF ACTIVE INGREDIENTS

(71) Applicants: Robert Davidson, Woodland Hills, CA (US); Anthony LaRosa, Woodland Hills, CA (US)

(72) Inventors: Robert Davidson, Woodland Hills, CA (US); Anthony LaRosa, Woodland Hills, CA (US)

(73) Assignee: Platform Delivery Technologies, Woodland Hills, CA (US)

(21) Appl. No.: 14/106,214

(22) Filed: Dec. 13, 2013

Related U.S. Application Data
(60) Provisional application No. 61/736,705, filed on Dec. 13, 2012.

(51) Int. Cl. A61M 31/00; A61K 47/44; A63B 71/08

(52) U.S. Cl. CPC A61M 31/002; A63B 71/085; A61K 47/44
USPC 128/861

(57) ABSTRACT

A mouthguard for delivering active ingredients to a user is provided. The mouthguard can include a U-shaped structure having an inner wall and an outer wall, the inner wall and outer wall connected to each other by a base defining a channel configured to receive upper teeth of a user, an active ingredient delivery area including a chamber having an inlet opening, the active ingredient delivery area including a perforated region including at least one aperture extending from an external surface to the chamber.

### CLAIM OF PRIORITY

This application claims the benefit of priority under 35 U.S.C. §119(e) of U.S. Provisional Patent Application Ser. No. 61/736,705, filed on Dec. 13, 2012, the benefit of priority of which is claimed hereby, and which is incorporated by reference herein in its entirety.

### TECHNICAL FIELD

The present disclosure relates to the field of dental appliances, and more particularly to mouthguards that deliver and facilitate active ingredients to a wearer thereof.

### BACKGROUND

Mouthguards are commonly worn by athletes to protect their teeth from the forces created by clenching teeth and to help reduce damage to teeth and surrounding soft tissue from activity-related traumas. Mouthguards can be molded to fit over a user's teeth of their upper jaw. Other mouthguards (e.g., stock mouthguards) are not molded to fit over a user's teeth. Once inserted into a user's mouth, the mouthguard forms a compliant layer between the user's upper teeth and lower mating mandibular teeth, or lower jaw teeth. All of a user's upper and lower teeth, are therefore insulated from making direct contact with each other. Mouthguards can be made of relatively soft elastomeric materials such as ethylene vinyl acetate and/or polyolefin elastomers that aid in minimizing dental traumas.

Mouthguards are often required for sports participants and are generally inexpensive to manufacture. Due to the required nature and ease of manufacture, mouthguards have become relatively ubiquitous.

### OVERVIEW

The present disclosure is directed to mouthguards that deliver at least one active ingredient to a wearer of the mouthguard. Using the mouthguard of the present disclosure can allow a user to receive beneficial ingredients while wearing the mouthguard. For example, if the mouthguard is worn during a sporting activity, active ingredients that are beneficial for sport participation can be delivered to the user.

The present inventors have recognized, among other things, that existing mouthguards do not provide active ingredients that can be beneficial to a user. For example, previous mouthguards have incorporated flavor into the mouthguards, for example, to mask the plastic taste of the mouthguards. However, while adding flavor to the mouthguard can increase the taste of a mouthguard, the flavor does not affect a user's performance or provide a physical benefit.

The present devices and methods provide a mouthguard that can deliver active ingredients to a user. The mouthguard can include a U-shaped structure having an inner wall and an outer wall, the inner wall and outer wall connected to each other by a base defining a channel configured to receive upper teeth of a user, an active ingredient delivery area including a chamber having an inlet opening, and a composition including at least one active ingredient positioned within the chamber.

This overview is intended to provide an overview of subject matter of the present patent application. It is not intended to provide an exclusive or exhaustive explanation of the disclosure. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of a mouthguard, as constructed in accordance with at least one example.

FIG. 2 illustrates a more detailed view of a portion of the mouthguard of FIG. 1 accepting an orally dissolving film cartridge, as constructed in accordance with at least one example.

FIG. 3 illustrates a cross-sectional view of the mouthguard of FIG. 1 along line 3-3, as constructed in accordance with at least one example.

FIG. 4 illustrates a perspective view of a portion of a mouthguard, as constructed in accordance with at least one example.

FIG. 5 illustrates a perspective view of a portion of a mouthguard, as constructed in accordance with at least one example.

FIG. 6 illustrates a cross-sectional view of the mouthguard of FIG. 5 along line 6-6, as constructed in accordance with at least one example.

FIG. 7 illustrates a perspective view of a mouthguard, as constructed in accordance with at least one example.

FIG. 8 illustrates a cross-sectional view of the mouthguard of FIG. 7 along line 7-7, as constructed in accordance with at least one example.

FIG. 9 illustrates a cross-sectional view of the mouthguard of FIG. 7 along line 7-7 accepting a composition, as constructed in accordance with at least one example.

FIG. 10 illustrates a perspective view of a mouthguard, as constructed in accordance with at least one example.

FIG. 11 illustrates a perspective view of the mouthguard of FIG. 10, as constructed in accordance with at least one example.

FIG. 12 illustrates a perspective view of a mouthguard, as constructed in accordance with at least one example.

FIG. 13 is a flowchart illustrating a method, as constructed in accordance with at least one example.

### DETAILED DESCRIPTION

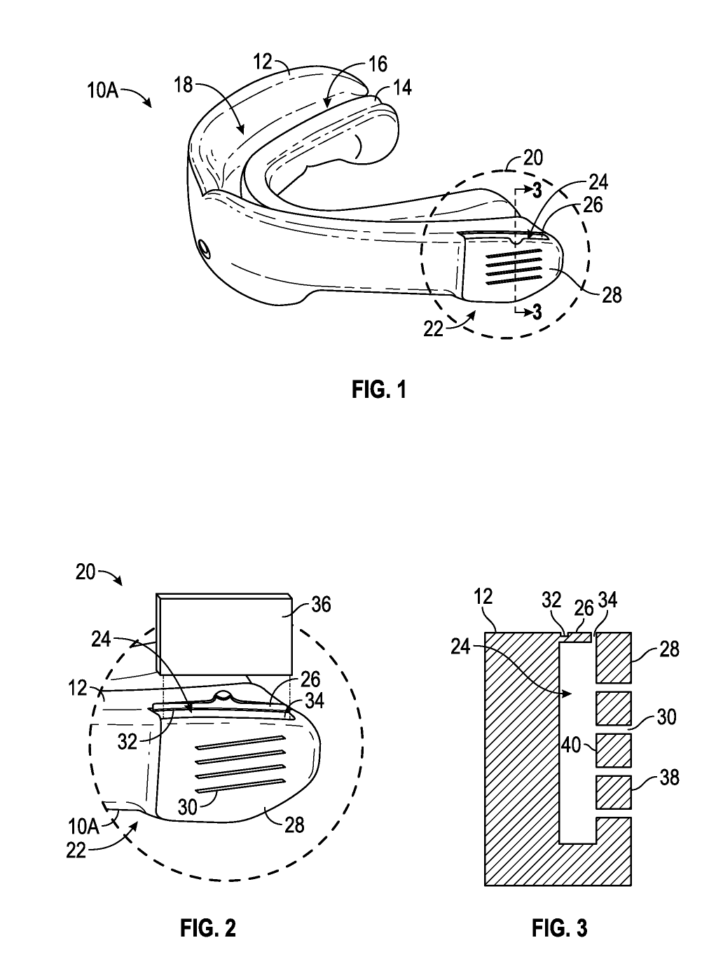

FIG. 1 illustrates a perspective view of a mouthguard 10, as constructed in accordance with at least one example. The mouthguard 10 can include provisions to disperse at least one active ingredient to a user of the mouthguard 10. As discussed herein, the term “active ingredient” can include prescription and over the counter active pharmaceutical ingredients (e.g., small molecules, macrocycles, 10 peptides, etc.), vitamins, nutraceuticals, supplements (e.g., dietary, nutritional, sports enhancement, and herbal), cosmetics, and biologicals.

In an example, the mouthguard can be disposable. In another example, the mouthguard can be reusable. When the mouthguard is reusable, the active ingredient delivery area can be refilled with active ingredient. The mouthguard can optionally be cleaned and disinfected between uses. When refilled with active ingredient, each subsequent active ingredient can independently be the same or different from previous active ingredients.

The active ingredient employed in the mouthguard can be in any suitable formulation dosage form. Suitable formulation dosage forms include, e.g., solid oral dosage forms (e.g., pill, tablet, capsule, powder, thin film, osmotic delivery system, and time release technology), and liquid dosage forms (e.g., elixir, emulsion, hydrogel, molecular encapsulation, ointment, paste, softgel, solution, suspension, syrup, tincture, and tisane).

The mouthguard 10A can include an outer wall 12 and an inner wall 14. The outer wall 12 and the inner wall 14 can be connected together by a base 16 forming a channel 18 (e.g., a semi-elliptical channel). The channel 18 can have a suitable size and dimension to substantially envelope the user's maxillary teeth to engage the upper lingual, occlusal, and incisal teeth surfaces. Different sizes and shapes of the mouthguard 10A are contemplated to accommodate a variety of mouth shapes and sizes. In an example, the channel 18 can be configured to receive upper teeth of a user. In an example, the mouthguard 10A can include another channel (not shown) opposite of the channel 18. The other channel can be configured to receive the mandibular teeth (e.g., lower teeth) of a user.

In an example, the mouthguard 10 can be a laminated mouthguard. For example, an outer frame of the laminated mouthguard can engage an elastomeric inner liner. Lamination can provide maximum protection with regard to the ability to absorb and disperse external forces. Additionally, lamination provides increased incisal area thickness and additional reinforcement between material layers.

In an example, the laminated mouthguard can be of a size and dimension so to fit in a user's mouth and allow the elastomeric inner liner to engage the surfaces of a majority or all of the user's upper, or maxillary, teeth. As discussed herein, the laminated mouthguard can be of a size and dimension to allow the elastomeric inner liner to engage the surfaces of a majority or all of the user's upper and lower teeth.

The mouthguard 10A can be semi-elliptical and the elastomeric inner liner can nest substantially within the semi-elliptical mouthguard. Additionally, the inner liner defines a semielliptical channel of a suitable size and dimension to substantially envelope the user's upper set of teeth to engage the upper lingual, occlusal, and incisal teeth surfaces. Different sizes and shapes of the mouthguard are contemplated to accommodate a variety of mouth shapes and sizes. Additionally, the mouthguard can be sized and shaped to substantially envelope the users's lower set of teeth.

In an example, the outer frame and inner liner are separate pieces of material that are bonded to each other. In one example, the outer frame is an overmolded layer bonded to the inner liner base. The overmolding is a flexible material, yet is of a higher durometer than the inner liner. In an alternative, a non-laminated example, the mouthguard is made from a single piece of material.

In an example, the mouthguard 10A can be constructed from elastomeric compounds. For example, polymers or copolymers can be used to form the mouthguard 10A. In an example, the mouthguard 10A can be formed of polyurethane, thermoplastic, Poly Vinyl acetate (PVA), and Ethyl Vinyl acetate (EVA). For example, the mouthguard 10A can be formed of thermoplastic selected from the group consisting of ethylene vinyl alcohol, ethylene vinyl acetate, urethane, styrene block copolymer, rubber, polystyrene, polybutadiene, polyisoprene, polyolefin, organopolysiloxane, alicyclic saturated hydrocarbon resin, polycaprolactone, polyethylene, unfilled polycarbonate, ester gum, polyethylenetetraphthalate, terpolymer, nylon, nylon copolymer, polyester, copolyester, or any combination of one or more thereof. The mouthguard 10A can be molded using a thermoforming process.

In an example, mouthguard 10A can include an active ingredient delivery area 22. As discussed herein, the active ingredient delivery area 22 can facilitate release (e.g., delivery) of at least one active ingredient to a user. For example, a composition including at least one active ingredient can be positioned within the chamber 24. The active ingredient delivery area 22 can include a chamber 24 having an inlet opening 34 (as shown in FIGS. 2 and 3). In an example, the active ingredient delivery area 22 can include a perforated region 28 that provides the composition in contact with a user's saliva. As shown in FIG. 1, the active ingredient delivery area 22 and chamber 24 are positioned along the outer wall 12 along a buccal surface (e.g., a surface that will contact a user's cheek) towards a distal end of the mouthguard 10A. However, it is contemplated that the active ingredient delivery area 22 can be positioned at various positions along the outer wall 12 or the inner wall 14. Additionally, the active ingredient delivery area 22 can be positioned along a buccal surface and/or a lingual surface. Further, while only one active ingredient delivery area 22 is shown in FIG. 1, the mouthguard 10A can include more than one active ingredient delivery area 22 along the outer and inner walls 12, 14 of the mouthguard 10A. Each active ingredient delivery area 22 can include the same composition or a different composition to delivery various active ingredients to a user.

FIG. 2 illustrates a more detailed view of a portion 20 of the mouthguard 10A of FIG. 1 accepting an orally dissolving film cartridge 36 (also referred to herein as “ODFC 36”), as constructed in accordance with at least one example. FIG. 3 illustrates a cross-sectional view of the mouthguard 10 of FIG. 1 along line 3-3, as constructed in accordance with at least one example. In an example, the ODFC 36 can be the composition positioned within the chamber 24. As discussed herein, the active ingredient delivery area 22 including the chamber 24 can facilitate release of at least one active ingredient.

As illustrated in the example of FIG. 2, the ODFC 36 can have a size and dimension to fit within the chamber 24. The ODFC 36 includes one or more active ingredients that can be released to the user over time. In an example, the ODCF 36 is about 5 millimeters (mm)×10 mm×1-2 mm in dimension, but any size and shape that accommodates a mouthguard is contemplated.

Referring to FIGS. 2 and 3, the perforated region 28 can provide the ODFC 36 in contact with a user's saliva. The perforated region 28 can include at least one aperture 30. In an example, the perforated region 28 includes a plurality of apertures 30. The at least one aperture 30 can extend from an external surface 38 of the first wall 12 to a surface 40 of the chamber 24. The at least one aperture 30 can allow a user's saliva to come into contact with the ODFC 36. As the ODFC 36 dissolves in the saliva, active ingredients are made available to the user. The size, shape, and composition of the ODFC 36 can dictate the time needed for the ODFC 36 to release substantially all the active ingredients therein.

As illustrated in the example shown in FIGS. 1-3, the ODFC 36 can be held inside the chamber 24 by a hinged door 26. The hinged door 26 can include a hinge 32. In an example, the hinge 32 can be a living hinge molded with the mouthguard 10A. However, other types of hinges can be used. The hinged door 26 can prevent the ODFC 36 from exiting the chamber 24 during use. For example, the hinged door 26 can maintain the ODFC 26 within the chamber 24 even if the user bites down on the mouthguard 10A or experiences impact to the mouth area such that the impact contacts the mouthguard 10A.

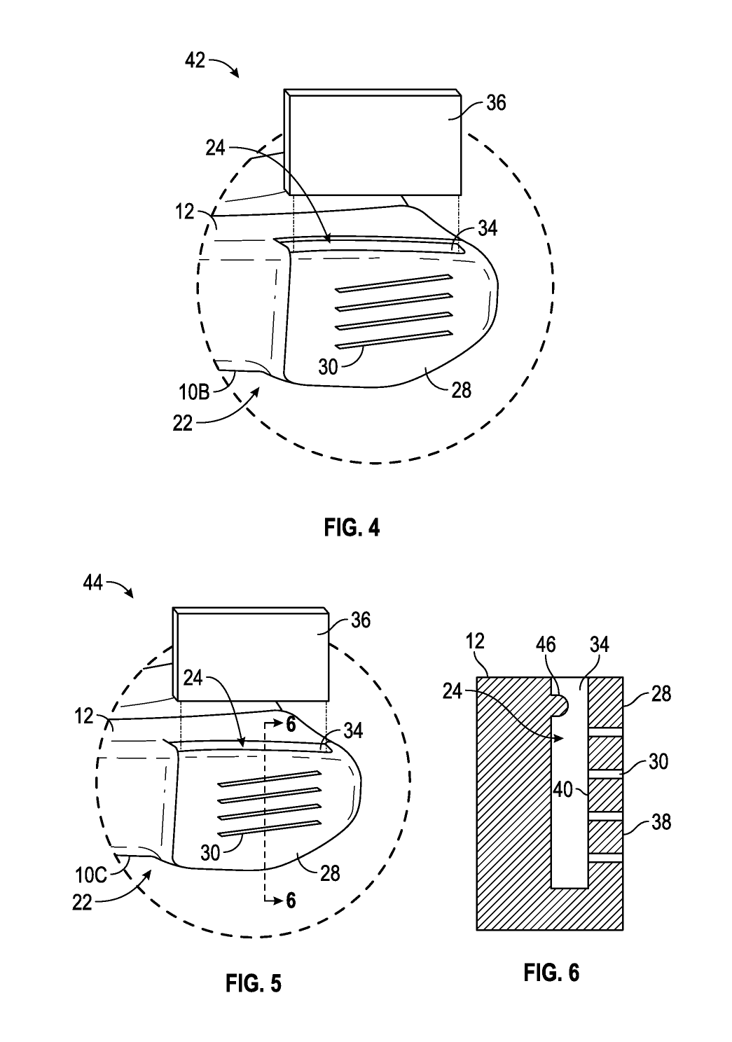

FIG. 4 illustrates a perspective view of a portion 42 of a mouthguard 10B, as constructed in accordance with at least one example. In an example, mouthguard 10B can be mouthguard 10A but include portion 42 instead of portion 20. The portion 42 can be similar to the portion 20 and include the active ingredient delivery area 22 including the chamber 24 having the inlet opening 34. The active ingredient delivery area 22 can include the perforated region 28 including at least one aperture 30. The difference between the portion 42 in FIG. 4 and the portion 22 in FIGS. 1-3, is that the portion 42 in FIG. 4 does not include the door 26 (as shown in FIGS. 1-3). In FIG. 4, the ODFC 36 can be held in the chamber 24 merely by friction or pressure exerted on the ODCF 36 by wall of the chamber 24. The friction or pressure exerted on the ODCF 36 is sufficient to prevent the ODCF 36 from exiting the chamber 24, for example, when a user bites down on the mouthguard 10B or when the user experiences impact that contacts the mouthguard 10B.

FIG. 5 illustrates a perspective view of a portion 44 of a mouthguard 10C, as constructed in accordance with at least one example. In an example, mouthguard 10C can be mouthguard 10A but include portion 44 instead of portion 20. For example, the portion 44 can be similar to the portion 20 and include the active ingredient delivery area 22 including the chamber 24 having the inlet opening 34. The active ingredient delivery area 22 can include the perforated region 28 including at least one aperture 30. The difference between the portion 44 in FIG. 5 and the portion 22 in FIGS. 1 and 2 is that the portion 44 in FIG. 5 does not include the door 26 (as shown in FIGS. 1-3) and can include a projection 46 (as shown in FIG. 6).

FIG. 6 illustrates a cross-sectional view of the mouthguard 10C of FIG. 5 along line 6-6, as constructed in accordance with at least one example. The projection 46 can extend into the chamber 24. In an example, the projection 46 can be molded with the mouthguard 10C. The projection 46 can be manually displaced by a user to allow installation of an ODFC 36 in the chamber 24. To displace the projection 46, the user may deform the mouthguard 10C to open the inlet opening 34 wide enough to accept an ODFC 36. Once the ODFC 36 is in the chamber 24, the projection 46 can acts as a detent that prevents the ODFC 36 from exiting the chamber 44.

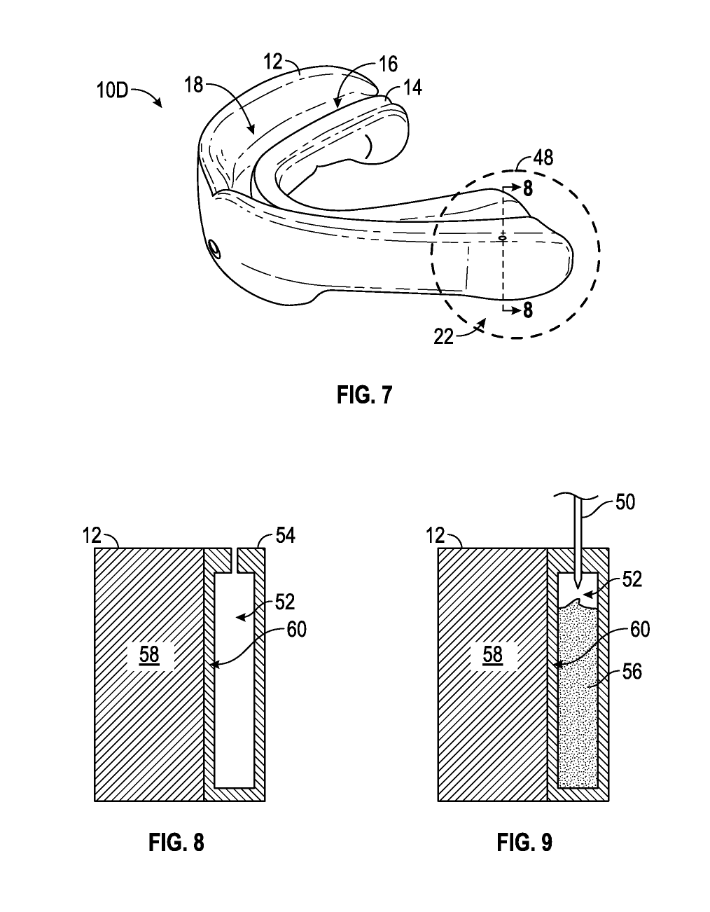

FIG. 7 illustrates a perspective view of a mouthguard 10D, as constructed in accordance with at least one example. FIG. 8 illustrates a cross-sectional view of the mouthguard 10D of FIG. 7 along line 7-7, as constructed in accordance with at least one example. In an example, mouthguard 10D can be mouthguard 10A but include portion 48 instead of portion 20. For example, mouthguard 10D can include the outer wall 12 and the inner wall 14, where the outer wall 12 and the inner wall 14 are connected together by the base 16 forming the channel 18. The portion 48 can include the active ingredient delivery area 22. In portion 48, the active ingredient delivery area 22 can include a first part 58 and a second part 60. The first part 58 can be formed of a first material and the second part 60 can be formed of a second material, different form the first material. In an example, the first material can be an impermeable material and the second material can be a permeable material. While all sides of the chamber 52 are shown to be formed of the second material, other examples can include were only the surface in contact with a buccal side (e.g., a user's cheek) can include the second material.

The first part 58 and the second part 60 can be coupled together, for example, by an adhesive, sutures, or other coupling mechanism. The second part 60 can define a chamber 52 configured to receive a composition (e.g., composition 56 in FIG. 9) including at least one active ingredient. The chamber 52 can have an opening 54. The opening 54, at a first position, can be substantially closed such that the composition is prevented form exiting the chamber 24. The opening 54 can be manually displaced from the first position (e.g., a substantially closed position) to a second position (e.g., an open position) to receive the composition 56.

FIG. 9 illustrates a cross-sectional view of the mouthguard 10D of FIG. 7 along line 7-7 accepting a composition, as constructed in accordance with at least one example. As shown in FIG. 9, a syringe 50 can be used to displace the opening 54 from the first position to the second position. Once inserted into the chamber 52, the syringe 50 can be activated to dispense the composition 56 within the chamber 52. For example, a plurality of syringes including the composition or a variety of compositions can be used with the mouthguard 10D. Once the composition 56 can be delivered to a user, the user can was and disinfect the mouthguard 10D and inject a composition that was the same or different from a previously injected composition. Additionally, a reusable syringe can be used to inject various compositions within the chamber 52.

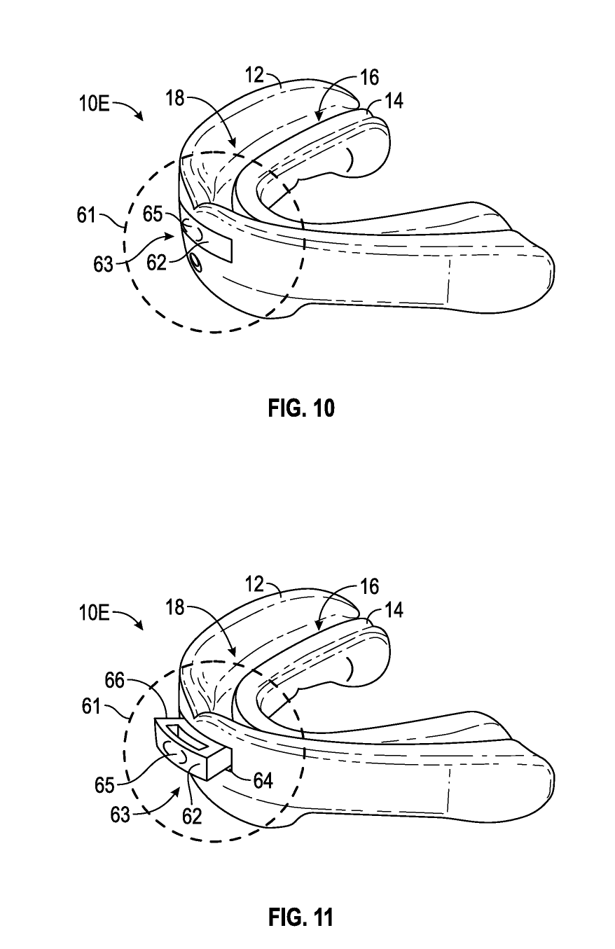

FIG. 10 illustrates a perspective view of a mouthguard 10E, as constructed in accordance with at least one example. In an example, mouthguard 10E can be mouthguard 10A but include portion 61 instead of portion 20. For example, mouthguard 10E can include the outer wall 12 and the inner wall 14, where the outer wall 12 and the inner wall 14 are connected together by the base 16 forming the channel 18. The portion 62 can include the active ingredient delivery area 63 including a chamber 64 (as shown in FIG. 11). In an example, the active ingredient delivery area 22 can include a tray 62 that is removable from the chamber 64 (as shown in FIG. 11) of the mouthguard 10E. The tray 62 can include an indentation 65 to facilitate removing the tray from the mouthguard 10E.

FIG. 11 illustrates a perspective view of the mouthguard 10E of FIG. 10, as constructed in accordance with at least one example. In FIG. 11, the tray 64 is partially removed from the chamber 64. In an example, the tray 62 can include a depression 66 that can be configured to receive a composition including the at least one active ingredient. Surfaces of the mouthguard 10E adjacent to the tray can be permeable, which can facilitate delivery of the at least one active ingredient. For example, once the tray 62 in reinserted into the mouthguard 10E, the composition positioned within the depression 66 of the tray can be delivered to the user. A surface of the tray 62, for example, a bottom surface can include a projection that can be configured to be received within a depression formed within the chamber 62. When the mouthguard 10E is inserted into the chamber 64, the projection can engage with the depression to prevent the tray 62 from exiting the chamber 64 while the user is wearing the mouthguard 10E. In an example, the friction created between the tray 62 and the chamber 64 of the mouthguard 10E can be sufficient to retain the tray 62 within the mouthguard 10E.

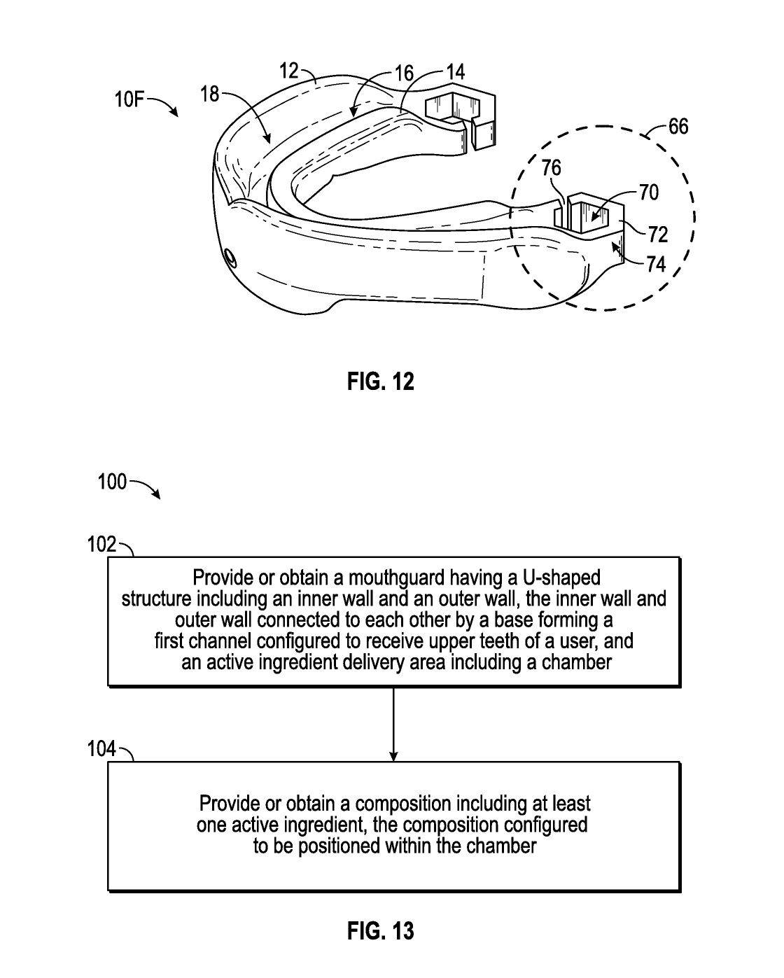

FIG. 12 illustrates a perspective view of a mouthguard 10F, as constructed in accordance with at least one example. In an example, mouthguard 10F can be mouthguard 10A but include portion 61 instead of portion 20. For example, mouthguard 10E can include the outer wall 12 and the inner wall 14, where the outer wall 12 and the inner wall 14 are connected together by the base 16 forming the channel 18. The portion 62 can include the active ingredient delivery area 74 including a chamber 70. The chamber 70 can be formed from a hook-like projection 72 extending from the distal end of the mouthguard 10F. The hook-like projection 72 can include opening 76, which can facilitate deformation of the chamber 70. For example, a user may deform the mouthguard 10F (e.g., deform the hook-like projection 72) to open the opening 76 wide enough to accept, for example, a capsule (e.g., a liquid filled capsule or pellet) including the composition having at least one active ingredient. Once the capsule is in the chamber 70, the user can release the hook-like depression 72, which can surround the capsule and prevent the capsule from exiting the chamber 70.

In another example, the hook-like projection 72 can be closed, thus forming a circular projection. A user can deform the projection 72 to open the chamber 70 large enough to receive the capsule. One the capsule is received, the user can release the projection 72 and the friction between the capsule and the projection 72 can maintain the capsule within the chamber 70. The capsule can dissolve over time while in a user's mouth.

In an example, the composition including the at least one active ingredient can be in the form of the ODFC 36, capsules, liquid filled capsules, gels, and liquids. In an example, the composition can be used to deliver the at least one active ingredient. As discussed herein, “active ingredients” can include prescription and over the counter active pharmaceutical ingredients (e.g., small molecules, macrocycles, 10 peptides, etc.), vitamins, nutraceuticals, supplements (e.g., dietary, nutritional, sports enhancement, and herbal), cosmetics, and biologicals. The composition (e.g., the ODFC 36) can be a composition for oral administration that dissolves in a mouth of a user.

In one example, the composition can include a sports enhancement supplement, including but not limited to electrolytes, energy promoting ingredients, and focus-improvement compounds. In one example, the composition includes electrolytes. The electrolytes of the composition can be selected, for example, from the group comprising of sodium, potassium, phosphate, bicarbonate, sulfate, chloride, calcium, and magnesium. For example, the composition may contain from about 1% by weight (w/w) to about 5% w/w potassium; from about 5% w/w to about 16% w/w sodium; from about 0.1% w/w to about 2% w/w magnesium; and from about 0.1% w/w to about 2% w/w calcium. In an example, the composition may contain about 2.75% w/w potassium chloride, about 13.75% w/w sodium chloride, about 0.5% w/w magnesium glycinate, and about 0.3% w/w calcium carbonate. In an example, the composition can include phosphate in concentrations varying from about 0.1% w/w to about 2% w/w.

In an example, the at least one active ingredient can be contained in a plurality of hydrophobic carriers dispersed throughout the composition (e.g., the ODFC 36). The hydrophobic carriers can be made of oil, for example, grapeseed oil.

In an example, the composition can include a phospholipid, an emulsifier, and a water soluble polymer. For example, the phospholipid can be hydroxylated lecithin, the emulsifier can be glycerin, and the water soluble polymer can be pectin. Flavoring agents can also be added to the composition, as well as sweetening agents.

Sugar substitutes, artificial and natural, are contemplated as the sweetening agents for the composition. In an example, the sweetening agents can include without limitation, stevia, aspartame, sucralose, neotame, acesulfame potassium, and saccharin. Natural sugar substitutes such as sorbitol and xylitol are also, without limitation, contemplated. In an example, the sweetener agent can be one or more of acesulfame potassium, sucrose, and sucralose.

In one example, of the present disclosure, the at least one active ingredient is at least partially contained in hydrophobic carriers that are dispersed in a water soluble polymer or mucoadhesive polymer. In an example, the hydrophobic carriers are either micelles, liposomes, or oil droplets in a colloidal suspension.

Some substances used to form micelles or liposomes include, but are not limited to, both natural and synthetic phosphatidyl-based substances (phospholipids) including lecithins (phosphatidylcholines), hydroxylated lecithin, polyethyleneglycol phospholipid, hydrogenated soy phosphatidylcholine, phosphatidic acid, phosphatidylglycerol, phosphatidylethanolamine, phosphatidylserine, sulfolipids such as sulfoquinovosyl distearoylglycerol, sulfates such as sodium lauryl sulfate, sulfonates such as dioctyl sodium sulfosuccinate, and carboxylates such as sodium deoxycholate, sodium stearate, and sodium oleate. The composition can include an amount of the phospholipid within a range of from about 0.0% to about 8.0% w/w and preferably in amounts of about 2% w/w to about 6% w/w, even more preferably about 3.5% w/w to about 4.5% w/w. In one example, the phospholipid is hydroxylated lecithin.

In an example where the composition comprises a colloidal suspension, the hydrophobic carriers can include lipophilic particles or droplets suspended in an aqueous medium. Such hydrophobic carriers can be formed from almond oil, argan oil, avocado oil, canola oil, cashew oil, castor oil, coconut oil, cod liver oil, colza oil, corn oil, cottonseed oil, fish oil, grapeseed oil, hazelnut oil, hemp oil, linseed oil (flaxseed oil), macadamia oil, manila oil, mongongo nut oil, mustard oil, olive oil, palm oil (palm kernel oil), peanut oil, pecan oil, perilla oil, pine nut oil, pistachio oil, poppy seed oil, pumpkin seed oil, grapeseed oil, rice bran oil, safflower oil, sesame oil, soybean oil, sunflower oil, tea seed oil, walnut oil, watermelon seed oil, and combinations thereof. In an example, the composition can include the hydrophobic carrier forming agent within a range of about 0.0% w/w to about 15% w/w, for example, about 2% to about 10% w/w such as 3.5% to about 4.5% w/w. In an example, the composition can include grapeseed oil due to its relatively low viscosity.

In an example, the composition can include one or more emulsifiers to prevent the hydrophobic carriers from agglomerating and settling into a continuous oil phase. The use of an emulsifier is more important in the colloidal suspensions. Suitable emulsifiers can include, but are not limited to, lecithin, hydroxylated lecithin, sodium stearyl lactylate, cetearyl alcohol, polysorbates, polyoxyethylene ethers, polyethylene glycol, anisolic compounds, and any conventional emulsifier. A preferred concentration range of emulsifier is approximately 0.0% w/w to about 20% w/w such as about 4% to about 14% w/w. In an example, the emulsifier is glycerin.

In an example, the composition can include water soluble polymers. Water soluble polymers can refer to any polymeric composition that is soluble in aqueous solution, and include, without limitation, cellulose derivatives such as hydroxyethylcellulose, methylcellulose, and hydroxypropylmethylcellulose, agarose, hyaluronan, acacia, amylase, casein, carboxymethyl cellulose, carboxyvinyl polymer, carrageenans, chitosan, collagen, dextrin, elsinan, gelatin, guar gum, gum Arabic, hydroxypropylated high amylase starch, levan, locust bean gum, methyl methacrylate copolymer, pectin, polyacrylic acid, polyethylene, polyvinyl alcohol, polyvinyl pyrrolidone, pullulan, sodium alginate, soy protein isolate, gum tragacanth, and whey. In an example, the composition can include the water soluble polymer within a range of about 0.0% to about 25% w/w, for example, 4% to about 20% w/w such as 9% w/w. In an example, the water soluble polymer is gelatin. In another example, the water soluble polymer is pectin.

In an example, the composition can include one or more mucoadhesive polymers. Mucoadhesive polymers refer to any polymer having a desirable in vivo mucosal absorption rate, level of safety, and rate of degradation. Examples of mucoadhesive polymers include, without limitation, alginate, chitosan, collagen, gelatin, hyaluronate, poly(ethyleneimine), poly(2-hydroxyethyl methacrylate), poly(acrylic acid), poly(ethylene oxide), and poly(L-lysine). The composition can include the mucoadhesive polymer within a range of from about 0.0% w/w to about 25% w/w, for example, about 8% w/w to about 20% w/w. In an example, the mucoadhesive polymer is gelatin.

In an example, the flavoring agents contemplated for the composition include, without limitation, almond, amaretto, amaretto nutty, anise, apple, apricot, banana creme, bavarian creme, bergamot, black walnut, blackberry, blueberry, brandy, bubble gum, butter, butter rum, butterscotch, cappuccino, caramel, champagne, cheesecake, cherry, cherry washington, chocolate, chocolate hazelnut, cinnamon, cinnamon roll, citrus blossom, clove, coconut, coffee, coffee keoke, coffee kona, cola, cotton candy, cranberry, cranraspberry, crème de menthe, eggnog, english toffee, ginger, grape, grapefruit, guava, hazelnut cream, honey, honeydew, horchata, horehound, hot chili, irish cream, key lime, lavender, lemon, lemonade, licorice, lime, mango, maple, marshmallow, melon, menthol eucalyptus, mint chocolate chip, mixed berry, mountain berry, nutmeg, orange brandy, orange cream, orange, peach, peanut butter, pear, pecan, peppermint, pina colada, pineapple, pistachio, plum, pomegranate, praline, pralines and cream, pumpkin, raspberry, red licorice, root beer, royal raspberry, salt water taffy, sassafras, spearmint, strawberry banana, strawberry, strawberry kiwi, tangerine, teaberry, tropical punch, tutti-frutti, vanilla butternut, vanilla, watermelon, and wintergreen. In an example, the flavoring agents can include cream and strawberry.

In an example, the composition can include preservatives. For example, sodium benzoate and potassium sorbate are preservatives that can be added for the purpose of keeping the composition fresh and to prevent bacteria from growing. Preservatives contemplated for the formulation include, without limitation, antimicrobial preservatives and antioxidants. Examples can include sorbic acid and its salts, benzoic acid and its salts, calcium propionate, sodium nitrite, sodium nitrate, sulfites, sulfur dioxide, sodium bisulfite, potassium hydrogen sulfite, disodium ethylenediaminetetraacetic acid (EDTA), Butylated hydroxyanisole, Butylated hydroxytoluene, tert-butylhydroquinone, propyl gallate, ethanol, and methylchloroisothiazolinone.

Food colorings such as FD&C Blue No. 1 (Brilliant Blue FCF), FD&C Blue No. 2 (Indigotine), FD&C Green No. 3 (Fast Green FCF), FD&C Red No. 3 (Erythrosine), FD&C Red No. 40 (Allura Red AC), FD&C Yellow No. 5 (Tartrazine), and FD&C Yellow No. 6 (Sunset Yellow FCF) are contemplated for addition to the composition to impart a desired color.

In an example, the composition can include vitamins. As used herein, the term “vitamin” refers to an organic compound required by an organism as a vital nutrient in limited amounts. An organic chemical compound (or related set of compounds) is called a vitamin when it cannot be 15 synthesized in sufficient quantities by an organism, and must be obtained from the diet. Thus, the term “vitamin” is conditional both on the circumstances and on the particular organism. For example, ascorbic acid (Vitamin C) is a vitamin for humans, but not for most other animals, and biotin and vitamin D are required in the human diet only in certain circumstances. Examples of human vitamins include Vitamin A (e.g., 20 retinol, retinal, and four carotenoids including beta carotene), Vitamin B1 (thiamine), Vitamin B2 (riboflavin), Vitamin B3 (e.g., niacin and niacinamide), Vitamin B5 (pantothenic acid), Vitamin B6 (e.g., pyridoxine, pyridoxamine, and pyridoxal), Vitamin B7 (biotin), Vitamin B9 (e.g., folic acid and folinic acid), Vitamin B12 (e.g., cyanocobalamin, hydroxocobalamin, and methylcobalamin), 25 Vitamin C (ascorbic acid), Vitamin D (cholecalciferol), Vitamin E (e.g., tocopherols and tocotrienols), and Vitamin K (e.g., phylloquinone, phytonadione, and menaquinones).

In an example, the composition can include nutraceuticals. As used herein, the term “nutraceutical” refers to a product isolated or purified from food that is generally sold in medicinal forms not usually associated with food. A nutraceutical is demonstrated to have a physiological benefit or provide protection against chronic disease. Such products can range from isolated nutrients, dietary supplements and specific diets to genetically engineered foods, and herbal products. Examples include antioxidants (e.g., pterostilbene from grapes and blueberries; resveratrol from red grape products; flavonoids inside citrus, tea, wine, and dark chocolate foods; and anthocyanins found in berries), substances believed to reduce hypercholesterolemia (e.g., soluble dietary fiber products, such as psyllium seed husk), substances believed to assist in cancer prevention (e.g., broccoli (sulforaphane) and fiddleheads (Matteuccia struthiopteris)), substances believed to improve arterial health (e.g., soy or clover (isoflavonoids)), substances believed to lower the risk of cardiovascular disease (e.g., alpha-linolenic acid from 10 flax or chia seeds, and omega 3 fatty acids in fish oil). Additional nutraceuticals can include, for example, botanical and herbal extracts such as ginseng, garlic oil, etc.

The composition can include a therapeutically effective amount of the one or more active ingredients. As used herein, “therapeutically effective amount” is intended to include an amount of a compound (e.g., active ingredient) described herein, or an amount of the combination of compounds (e.g., active ingredients) described herein, for example, to treat or prevent the disease or disorder, or to treat the symptoms of the disease or disorder, in a host. In an example, the combination of compounds can be a synergistic combination. Synergy, as described for example by Chou and Talalay, Adv. Enzyme Regul., 22:27 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As discussed herein, the at least one active ingredient can treat or prevent the disease or disorder, or to treat the symptoms of the disease or disorder, in a host. As used herein, “treating” or “treat” includes: (i) preventing a pathologic condition from occurring (e.g. prophylaxis); (ii) inhibiting the pathologic condition or arresting its development; (iii) relieving the pathologic condition; and/or (iv) diminishing symptoms associated with the pathologic condition. The composition including the at least one active ingredient can be administered, e.g., to a human patient in need of a treatment of a disease or disorder. Selection of the active ingredient(s) within the composition described herein will be dependent upon the disease or disorder to be treated. The Physician's Desk Reference (2010 Edition) provides a description of the diseases or disorders that specific active ingredients have been approved for by the U.S. FDA, in the marketing and sale of the product within the United States. As such, a skilled artisan can look to such references for guidance in the selection of the active ingredient(s) to be present within the composition, based upon the treatment of the specific disease or disorder of particular interest. The at least one active ingredients can be pharmaceutically acceptable. The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

In an example, the at least one active ingredient can be present in any suitable and appropriate amount, depending upon the desired dosing. For example, in a 100 milligram (mg) ODFC 36, for example, the at least one active ingredient can be present in an amount of about 0.01 mg to about 60 mg, for example, about 0.1 mg to about 50 mg such as about 0.5 mg to about 40 mg.

In an example, the composition can include pharmaceutical ingredients (e.g., active pharmaceutical ingredients (APIs)). APIs can include, but are not limited to, ace-inhibitors, anti-Alzheimer's agents, antianginal drugs, anti-arrhythmias, antiasthmatics, anti-cholesterolemics, analgesics, anesthetics, anti-convulsants, antidepressants, anti-diabetic agents, anti-diarrhea preparations, antidotes, anti-emetics, anti-histamines, anti-hypertensive drugs, anti-inflammatory agents, anti-lipid agents, anti-manics, anti-migraines, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, antiparkinsonian agents, anti-rheumatic agents, anxiolytics, anti-psychotics, appetite stimulants, biological response modifiers, blood modifiers, bone metabolism regulators, bronchodilators, cardiovascular agents, central nervous system stimulates, cholinesterase inhibitors, contraceptives, decongestants, dietary supplements, dopamine receptor agonists, endometriosis management agents, enzymes, erectile dysfunction agents, fertility agents, gastrointestinal agents, H2-antagonists, homeopathic remedies, hormones, hypercalcemia and hypocalcemia management agents, immunomodulators, immunosuppressives, migraine preparations, motion sickness treatments, muscle relaxants, non-steroidal anti-inflammatories (NSAID's), obesity management agents, osteoporosis preparations, oxytocics, parasympatholytics, parasympathomimetics, prostaglandins, psychotherapeutic agents, respiratory agents, sedatives, serotonin 5-HT3 receptor antagonists, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anticonvulsants, neuromuscular drugs, hyper- and hypo-glycemic agents, thyroid and anti-thyroid preparations, diuretics, anti-spasmodics, anti-obesity drugs, erythropoietic drugs, anti-asthmatics, cough suppressants, mucolytics, DNA and genetic modifying drugs, and combinations thereof.

In an example, the composition can include medicating active ingredients. Medicating active ingredients can include, but are not limited to, include antacids, H2-antagonists, and analgesics. For example, antacid dosages can be prepared using the ingredients calcium carbonate alone or in combination with magnesium hydroxide, and/or aluminum hydroxide. Moreover, antacids can be used in combination with H2-antagonists. Analgesics include opiates and opiate derivatives, such as oxycodone (available as Oxycontin®), ibuprofen, aspirin (available as Bayer®), acetaminophen, and combinations thereof that may optionally include caffeine. Other active ingredients that may be used in the present disclosure include anti-diarrheals such as Imodium AD®, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present disclosure.

In an example, the composition can include at least one active ingredient selected from adrenergic agonists such as clonidine; anxiolytics such as alprazolam (available as Xanax®); anti-psychotics such as clozapine (available as Clozaril®) and haloperidol (available as Haldol®); non-steroidal anti-inflammatories (NSAID's) such as dicyclofenac (available as Voltaren®) and etodolac (available as Lodine®), anti-histamines such as loratadine (available as Claritin®), astemizole (available as Hismanal®), nabumetone (available as Relafen®), fexofenadine (available as Allegra®), and clemastine (available as Tavist®); anti-emetics such as granisetron hydrochloride (available as Kytril®), serotonin 5-HT3 receptor antagonists such as ondansetron (available as Zofran®) and nabilone (available as Cesamet™); bronchodilators such as salbutamol (aka albuterol, available as Ventolin®), albuterol sulfate (available as Proventil®); anti-depressants such as fluoxetine hydrochloride (available as Prozac®), sertraline hydrochloride (available as Zoloft®), and paroxetine hydrochloride (available as Paxil®); anti-migraines such as sumatriptan (available as Imigran®), ACE-inhibitors such as enalapril (available as Vasotec®), captopril (available as Capoten®) and lisinopril (available as Prinivil® and Zestril®); anti-Alzheimer's agents, such as nicergoline; calcium channel blocker (CCB) such as nifedipine (available as Procardia® and Adalat®), and verapamil hydrochloride (available as Calan®); opioid analgesics such as fentanyl (available as Sublimaze®), alfentanil, sufentanil, remifentanil, carfentanil, and lofentanil; cough suppressants such as dextromethorphan; local anesthetics such as benzocaine (available as Cepacol® and Anbesol®); peptide hormones such as insulin; oral contraceptives such as estrogen (estradiol) and a progestogen (progestin); vaccines such as killed vaccines (e.g., influenza vaccine, cholera vaccine, bubonic plague vaccine, polio vaccine, hepatitis A vaccine, and rabies vaccine); attenuated vaccines (e.g., yellow fever, measles, rubella, and mumps); toxoid vaccines (e.g., tetanus and diphtheria); subunit vaccines (e.g., subunit vaccine against Hepatitis B virus, viruslike particle (VLP) vaccine against human papillomavirus, and the hemagglutinin and neuraminidase subunits of the influenza virus); fluoridating agents such as sodium fluoride, sodium monofluorophosphate (MFP) and stannous fluoride; stimulants such as caffeine, theobromine, theophylline, yohimbine, and nicotine; energy boosters such as methylxanthines (e.g., caffeine), B vitamins (e.g., Vitamin B12), herbs, guarana, verba mate, açai, taurine, various forms of ginseng, maltodextrin, inositol, carnitine, creatine, glucuronolactone, ginkgo biloba, bitter orange extract, coenzyme Q10, amino acids (e.g., L-carnitine), bee pollen, royal jelly, green tea extract, spirulina, gotu kola, and glucose; opioid antidiarrheals such as loperamide (available as Imodium®); sports supplements such as fish oil, dietary protein, creatine, caffeine, glutamine, essential fatty acids (e.g., (alpha-linolenic acid and linoleic acid), prohormones (e.g., chrysin and 4-androstene-3,6,17-trione), and testosterone boosters (e.g., Fenugreek, Eurycoma longifolia, D-Aspartic acid, Boron, L-Carnitine and Tribulus terrestris); analgesics such as non-steroidal antiinflammatory drugs (NSAIDs); COX-2 inhibitors such as rofecoxib, celecoxib and etoricoxib; opiates such as morphine, diacetylmorphine, codeine, oxycodone, hydrocodone, dihydromorphine, pentazocine, butorphanol, and pethidine; dietary supplements such as melatonin (N-acetyl-5-methoxytryptamine), vitamins, minerals, fiber, fatty acids, and amino acids; electrolytes such as sodium (Na+), potassium (K+), calcium (Ca2+), magnesium (Mg2+), chloride (Cl−), hydrogen phosphate (HPO42−), and hydrogen carbonate (HCO3−); artificial sweeteners and natural sweeteners.

The at least one active ingredient can include erectile dysfunction therapies include, e.g., drugs for facilitating blood flow to the penis, and for effecting autonomic nervous activities, such as increasing parasympathetic (cholinergic) and decreasing sympathetic (adrenergic) activities. In an example, useful non-limiting drugs can include sildenafil and pharmaceutically acceptable salts thereof, such as Viagra®, tadalafil, such as Clalis®, vardenafil and pharmaceutically acceptable salts thereof, such as Levitra®, apomorphine and pharmaceutically acceptable salts thereof, such as Uprima®, yohimbine hydrochloride such as Aphrodyne®, and alprostadil such as Caverject®. The popular H2-antagonists which are contemplated for use in the present disclosure include cimetidine, ranitidine hydrochloride, famotidine, nizatidine, ebrotidine, mifentidine, roxatidine, pisatidine, and aceroxatidine.

Specific suitable active ingredients can include ondansetron (available as Zuplenz® and Zofran®), diphenhydramine (available as Benadryl®); simethicone (available as Gas-X®); melatonin (available as Melatonin PM®); benzocaine (available as Orajel®); buprenorphine and naloxone (available as Suboxone®); buprenorphine (available as Subutex®); phenylephrine or pseudoephedrine (available as Sudafed®); acetaminophen, chlorpheniramine maleate, dextromethorphan hydrobromide, and pseudoephedrine hydrochloride (available as Theraflu®); and paracetamol and phenylephrine hydrochloride (available as Lemsip®).

Because the compositions can be administered to a subject in need thereof, one having ordinary skill in the art will recognize that the compositions can further contain substances used for the preparation of a final dosage form as is readily understood in the pharmaceutical and nutraceutical arts. Examples of these substances include one or more excipients, diluents, disintegrants, emulsifiers, solvents, processing aids, buffering agents, colorants, flavorings, solvents, coating agents, binders, carriers, glidants, lubricants, granulating agents, gelling agents, polishing agents, suspending agent, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, essential oils, emollients, dissolution enhancers, dispersing agents, or combinations thereof.

In another example of a composition for an ODFC 16, administration of sleep enhancement supplements via a mouthguard indicated for nighttime use is contemplated.

As discussed herein, besides the ODFC 36, tablets, capsules, and liquid filled capsules may be configured to fit within the chamber to sustainably deliver active ingredients to a user. Additionally, the chamber could be permeable so as to allow for a syringe (or any related form of injection device) to fill the chamber within the device for purposes of delivery of an active ingredient to a user.

FIG. 13 is a flowchart illustrating a method, as constructed in accordance with at least one example. The method 100, at step 102, can include providing or obtaining a mouthguard having a U-shaped structure including an inner wall and an outer wall, the inner wall and outer wall connected to each other by a base forming a first channel configured to receive upper teeth of a user and an active ingredient delivery area including a chamber. The method 100, at step 104 can include providing or obtaining a composition including at least one active ingredient, the composition configured to be positioned within the chamber.

In an example, the method 100 can include where providing or obtaining the composition including the at least one active ingredient includes providing or forming the composition including a sport enhancement supplement, the sport enhancement supplement chosen from electrolytes, energy promoting ingredients, and focus-improvement compounds.

The present disclosure has been described hereinabove with reference to the accompanying drawings, in which preferred embodiments of the disclosure are shown. Unless otherwise defined, all technical terms used herein are intended to have the same meaning as commonly understood in the art to which this disclosure pertains and at the time of its filing. Although various methods and materials similar or equivalent to those described herein can be used in the practice or testing of the present disclosure, only some of the suitable methods and materials are described. The skilled should understand that the methods and materials used and described are examples and may not be the only ones suitable for use in the disclosure.

Accordingly, this disclosure may be embodied in many different forms and should not be construed as limited to the illustrated embodiments set forth herein. Rather, these illustrated embodiments are provided so that this disclosure will be thorough, complete, and will fully convey the scope of the disclosure to those skilled in the art. Therefore, in the specification set forth above there have been disclosed typical preferred embodiments of the disclosure, and although specific terms are employed, the terms are used in a descriptive sense only and not for purposes of limitation. The disclosure has been described in some detail, but it will be apparent that various modifications and changes can be made within the spirit and scope of the disclosure as described in the foregoing specification.

In this application, reference is made to particular features (including method steps) of the present disclosure. It is to be understood that the present disclosure includes all possible combinations of such particular features, regardless of whether a combination is explicitly described. For example, where a particular feature is disclosed in the context of a particular aspect or example of the present disclosure, that feature can also be used, to the extent possible, in combination with and/or in the context of other particular aspects and examples of the present disclosure, and in the disclosure generally.

The term “comprises” is used herein to mean that other features or steps are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

This disclosure may be embodied in many different forms and should not be construed as limited to the embodiments set forth herein. Rather, these embodiments are provided so that this disclosure will be thorough and complete, and will convey the scope of the disclosure to those skilled in the art.

Embodiments of the disclosure are described herein in connection with protective mouthguards having the ability to dispense an active ingredient. In this context, the drawings illustrate these embodiments by showing athletic mouthguards. It is to be understood, however, that the disclosure is not limited to the specific size or shape mouthguard that is described. The disclosure may be adapted as desired for use with any size or shape dental appliance.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the disclosure should be determined with reference to the appended claims, along with the full scope of equivalents to which such claims are entitled.

Specific Examples 1 to ______ provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.

### ENUMERATED EXAMPLES

Example 1 includes subject matter directed to a mouthguard device. The mouthguard device can include a U-shaped structure having an inner wall and an outer wall, the inner wall and outer wall connected to each other by a base defining a channel configured to receive upper teeth of a user, an active ingredient delivery area including a chamber having an inlet opening, and a composition including at least one active ingredient positioned within the chamber.

In Example 2, the subject matter of Example 1 can optionally include where the active ingredient delivery area is positioned within the outer wall.

In Example 3, the subject matter of one or any combination of Examples 1-2 can optionally include where the active ingredient delivery area includes a perforated region including at least one aperture.

In Example 4, the subject matter of one or any combination of Examples 1-3 optionally include where the active ingredient delivery area includes a hinged door positioned at the inlet opening of the chamber.

In Example 5, the subject matter of one or any combination of Examples 1-4 can optionally include where the active ingredient delivery area includes a projection extending into the chamber.

In Example 6, the subject matter of one or any combination of Examples 1-5 optionally includes where at least a portion of the active ingredient delivery area includes a permeable material.

In Example 7, the subject matter of one or any combination of Examples 1-6 can optionally include where the inlet opening of the chamber is closed at a first position and is open in a second position.

In Example 8, the subject matter of one or any combination of Examples 1-7 can optionally include where the active ingredient delivery area includes a tray positioned within the chamber, the tray being removable from the chamber.

In Example 9, the subject matter of one or any combination of Examples 1-8 can optionally include where the tray includes a depression configured to receive the composition

Example 10, the subject matter of one or any combination of Examples 1-9 can optionally include where the composition is in the form of at least one of an orally dissolving film cartridge, a tablet, a capsule, a liquid filled capsule, a liquid, and a gel.

In Example 11, the subject matter of one or any combination of Examples 1-10 can optionally include where the composition is an orally dissolving film cartridge.

In Example 12, the subject matter of one or any combination of Examples 1-11 can optionally include where the at least one active ingredient is chosen from pharmaceutical ingredients, vitamins, nutraceuticals, supplements, cosmetics, and biologicals.

In Example 13, the subject matter of one or any combination of Examples 1-12 can optionally include where the at least one active ingredient is a sport enhancement supplement.

In Example 14, the subject matter of one or any combination of Examples 1-13 can optionally include where the sport enhancement supplement is chosen from electrolytes, energy promoting ingredients, and focus-improvement compounds.

In Example 15, the subject matter of one or any combination of Examples 1-14 can optionally include where the at least one active ingredient is an electrolyte chosen from sodium, potassium, phosphate, bicarbonate, sulfate, chloride, calcium, and magnesium

In Example 16, the subject matter of one or any combination of Examples 1-15 can optionally include where the composition includes a hydrophobic carrier.

In Example 17, the subject matter of one or any combination of Examples 1-16 can optionally include where the hydrophobic carrier is chosen from micelles, liposomes, and oil droplets in a colloidal suspension

In Example 18, the subject matter of one or any combination of Examples 1-17 can optionally include where the composition includes a phospholipid, an emulsifier, and a water soluble polymer.

Example 19 includes subject matter directed toward a mouthguard system. The mouthguard system includes a U-shaped structure having an inner wall and an outer wall, the inner wall and outer wall connected to each other by a base forming a channel configured to receive upper teeth of a user, and an active ingredient delivery area including a chamber having an inlet opening, and a plurality of orally dissolving film cartridges configured to be positioned within the chamber.

In Example 20, the subject matter of one or any combination of Examples 1-19 can optionally include where a first orally dissolving film cartridge of the plurality of orally dissolving film cartridges is different from a second orally dissolving film cartridge of the plurality of orally dissolving film cartridges.

In Example 21, the subject matter of one or any combination of Examples 1-20 can optionally include where the at least one active ingredient is chosen from pharmaceutical ingredients, vitamins, nutraceuticals, supplements, cosmetics, and biologicals.

In Example 22, the subject matter of one or any combination of Examples 1-21 can optionally include where the at least one active ingredient is a sport enhancement supplement, the sport enhancement supplement is chosen from electrolytes, energy promoting ingredients, and focus-improvement compounds.

Example 23 includes subject matter including a method. The method can include providing or obtaining a mouthguard having a U-shaped structure including an inner wall and an outer wall, the inner wall and outer wall connected to each other by a base forming a first channel configured to receive upper teeth of a user, and an active ingredient delivery area including a chamber, and providing or obtaining a composition including at least one active ingredient, the composition configured to be positioned within the chamber.

In Example 23, the subject matter of one or any combination of Examples 1-22 can optionally include where providing or obtaining the composition including the at least one active ingredient includes providing or forming the composition including a sport enhancement supplement, the sport enhancement supplement chosen from electrolytes, energy promoting ingredients, and focus-improvement compounds.

What is claimed is:

1. A mouthguard device, comprising:
a U-shaped structure having an inner wall and an outer wall, the inner wall and outer wall connected to each other by a base defining a channel configured to receive upper teeth of a user;
an active ingredient delivery area including a chamber having an inlet opening, the active ingredient delivery area including a perforated region including at least one aperture extending from an external surface to the chamber; and
a composition including at least one active ingredient positioned within the chamber.

2. The mouthguard device of claim 1, wherein the active ingredient delivery area is positioned within the outer wall.

3. The mouthguard device of claim 1, wherein the active ingredient delivery area is positioned along a buccal surface of the outer wall.

4. The mouthguard device of claim 1, wherein the active ingredient delivery area includes a hinged door positioned at the inlet opening of the chamber.

5. The mouthguard device of claim 1, wherein the active ingredient delivery area includes a projection extending into the chamber.

6. The mouthguard device of claim 1, wherein at least a portion of the active ingredient delivery area includes a permeable material.

7. The mouthguard device of claim 1, wherein the composition is an orally dissolving film cartridge.

8. The mouthguard device of claim 1, wherein the at least one active ingredient is chosen from pharmaceutical ingredients, vitamins, nutraceuticals, supplements, cosmetics, and biologicals.

9. The mouthguard device of claim 1, wherein the at least one active ingredient is a sport enhancement supplement.

10. The mouthguard device of claim 9, wherein the sport enhancement supplement is chosen from electrolytes, energy promoting ingredients, and focus-improvement compounds.

11. The mouthguard device of claim 1, wherein the at least one active ingredient is an electrolyte chosen from sodium, potassium, phosphate, bicarbonate, sulfate, chloride, calcium, and magnesium

12. The mouthguard device of claim 1, wherein the composition includes a hydrophobic carrier.

13. The mouthguard device of claim 12, wherein the hydrophobic carrier is chosen from micelles, liposomes, and oil droplets in a colloidal suspension.

14. The mouthguard device of claim 1, wherein the composition includes a phospholipid, an emulsifier, and a water soluble polymer.

15. A mouthguard system, comprising:
a mouthguard, including:
a U-shaped structure having an inner wall and an outer wall, the inner wall and outer wall connected to each other by a base forming a channel configured to receive upper teeth of a user; and
an active ingredient delivery area including a chamber having an inlet opening, the active ingredient delivery area including a perforated region including at least one aperture extending from an external surface to the chamber; and
a plurality of orally dissolving film cartridges configured to be positioned within the chamber.

16. The mouthguard system of claim 15, wherein a first orally dissolving film cartridge of the plurality of orally dissolving film cartridges is different from a second orally dissolving film cartridge of the plurality of orally dissolving film cartridges.

17. The mouthguard system of claim 15, wherein the at least one active ingredient is chosen from pharmaceutical ingredients, vitamins, nutraceuticals, supplements, cosmetics, and biologicals.

18. The mouthguard system of claim 15, wherein the at least one active ingredient is a sport enhancement supplement, the sport enhancement supplement is chosen from electrolytes, energy promoting ingredients, and focus-improvement compounds.

19. A method comprising:
providing or obtaining a mouthguard having a U-shaped structure including an inner wall and an outer wall, the inner wall and outer wall connected to each other by a base forming a first channel configured to receive upper teeth of a user, and an active ingredient delivery area including a chamber having an inlet opening, the active ingredient delivery area including a perforated region including at least one aperture extending from an external surface to the chamber; and
providing or obtaining a composition including at least one active ingredient, the composition configured to be positioned within the chamber.

20. The method of claim 19, wherein providing or obtaining the composition including the at least one active ingredient includes providing or forming the composition including a sport enhancement supplement, the sport enhancement supplement chosen from electrolytes, energy promoting ingredients, and focus-improvement compounds.
